(19) United States
(12) Patent Application Publication
(10) Pub. No.: US 2012/0271245 A1
(43) Pub. Date: Oct. 25, 2012

(54) MALLEABLE STOPPER FOR A SYRINGE

(76) Inventor: LEONARD ACHAN, JR., New York, NY (US)

(21) Appl. No.: 13/453,873

(22) Filed: Apr. 23, 2012

Related U.S. Application Data
(60) Provisional application No. 61/477,739, filed on Apr. 21, 2011.

(51) Int. Cl. A61M 5/315

(52) U.S. Cl. 604/236

(57) ABSTRACT

A syringe stopper for use in a syringe having a substantially cylindrical syringe barrel and a piston-like plunger rod for sliding inside the syringe barrel in a fluid-tight engagement. A malleable stopper is located on a distal end of the plunger rod and protrudes outwardly therefrom. The malleable stopper changes shape in response to a pressure applied thereon.

### RELATED APPLICATION

This application claims the benefit of U.S. Provisional Application No. 61/477,739 filed Apr. 21, 2011, which is incorporated herein by reference in its entirety.

### FIELD OF INVENTION

The present invention relates to syringes and syringe stoppers, and more particularly to a malleable syringe stopper which helps provide for a more complete expulsion of the contents of the syringe at the time of drug delivery.

### BACKGROUND OF THE INVENTION

Hypodermic syringes include an elongate barrel having opposed proximal and distal ends and a chamber therebetween for receiving a fluid. A passageway extends through the distal end of the syringe barrel and communicates with the chamber. The distal end of the syringe barrel is connected to a needle cannula for delivering fluid from the chamber and passageway. The proximal end of the syringe barrel slidably receives a plunger rod and stopper assembly, such that force applied to the plunger rod urges the stopper along the barrel to drive liquid from the chamber through the needle cannula.

A problem with some prior art hypodermic syringes involves the amount of liquid which can remain in the barrel after the stopper is advanced the full length of the barrel during the injection process. There remains in every syringe a certain residual volume, no matter how small, which is trapped between the end of the stopper and the needle tip, including the volume of the needle cannula and the volume of the extreme distal end of the syringe barrel. For very expensive liquid medications and medications which require very precise dose delivery it may be necessary to overfill the barrel for the amount of the dead space so that the dose delivered will be the dose desired and the additional medication will be disposed of with the used syringe. Over many of syringes the amount of unused medication can become substantial.

One way to minimize dead space is to carefully control the dimensions of the inside of the syringe barrel and to conform the distal end of the stopper as closely as possible to the syringe barrel dimensions. This solution is not a complete answer because, especially in the case of glass syringe barrels, the interior shape of the barrel, as it transitions between the bore of the chamber to the needle holding tip cannot be as precisely controlled as an injection molded plastic barrel. Further, when the angle of the stopper tip is designed to conform with the interior angle of the barrel, variations in dimensions experienced in the manufacturing process can result in angular variations between the surfaces of the stopper and the barrel interior wherein the distal portion of the stopper is capable of occluding the passageway at the distal end of the barrel and trapping fluid in the barrel behind the distal end of the stopper.

Accordingly, the claimed invention proceeds upon the desirability of providing a syringe stopper, preferably a malleable syringe stopper, which minimizes dead space to assure that liquid medication is not trapped in the barrel when the stopper occludes or blocks off the exit passageway in the barrel.

### SUMMARY OF THE INVENTION

Therefore, an object of the claimed invention is to provide a malleable (or shape changing) syringe stopper which minimizes any dead space when the syringe stopper is at the fully advanced position to maximize the expulsion of the liquid medication from the syringe barrel.

Another object of the claimed invention is to provide aforesaid syringe stopper which extends to the syringe tip.

The syringe stopper of the claimed invention, for use in a syringe barrel having a substantially cylindrical body, comprises a piston-like stopper body for slidable fluid-tight engagement inside the barrel of the syringe. The body includes a distal end, a proximal end and a longitudinal axis therethrough. A distally directed, generally conically-shaped malleable protrusion is provided on the distal end of the stopper body. The conically-shaped protrusion is malleable to fill-up any dead space in the syringe barrel, including the syringe tip, when the stopper body is at the distal end of the barrel (or at the fully advanced position) to assure that liquid mediation is not trapped in the syringe barrel.

The stopper can be combined with a syringe barrel having an open proximal end, a distal end and a cylindrical body portion therebetween which defines a chamber for retaining liquid. The stopper, in the combination, is positioned in fluid-tight engagement inside the barrel so the distal end of the stopper faces the distal end of the barrel. The distal end of the barrel includes an aperture therethrough in fluid communication with the chamber. In some applications, the proximal end of the stopper may be connected to the distal end of a rigid elongate plunger rod.

Various other objects, advantages, and features of the claimed invention will become readily apparent from the ensuing detailed description, and the novel features will be particularly pointed out in the appended claims

### BRIEF DESCRIPTION OF THE DRAWINGS

The following detailed description, given by way of example, and not intended to limit the claimed invention solely thereto, will best be understood in conjunction with the accompanying drawings in which:

FIG. 1 is a side partial cross-sectional view of a syringe assembly utilizing the stopper in accordance with an exemplary embodiment of the claimed invention; and

FIG. 2 is an enlarged partial cross-sectional view of the syringe of FIG. 1 showing the stopper in a fully advanced position expelling the last portion of the medication.

### DETAILED DESCRIPTION

The term “distal end” as used herein is intended to refer to the end of the syringe is closer to the syringe needle and the term “proximal end” is intended to refer to the end of the syringe closer to the holder of the syringe.

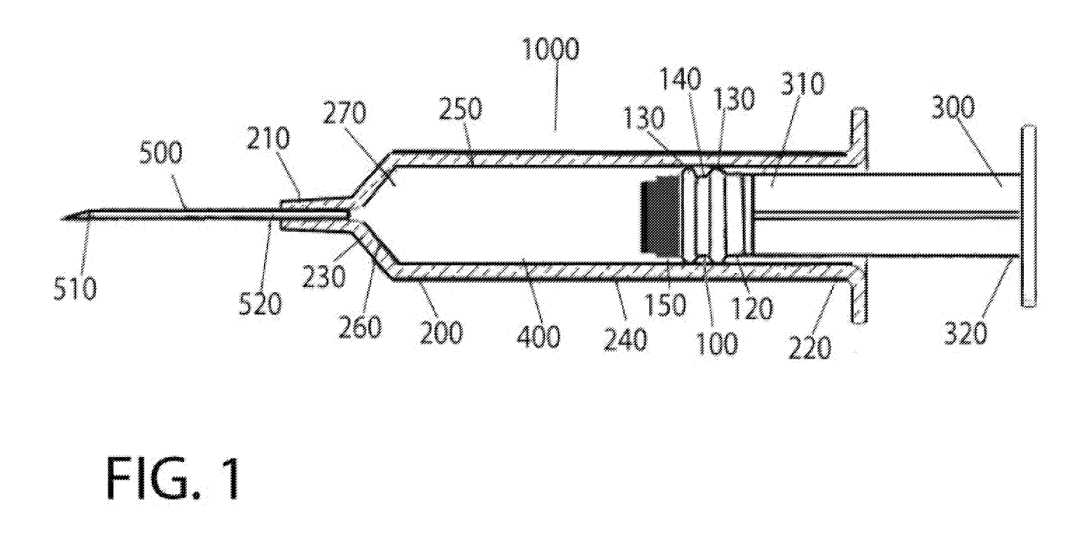

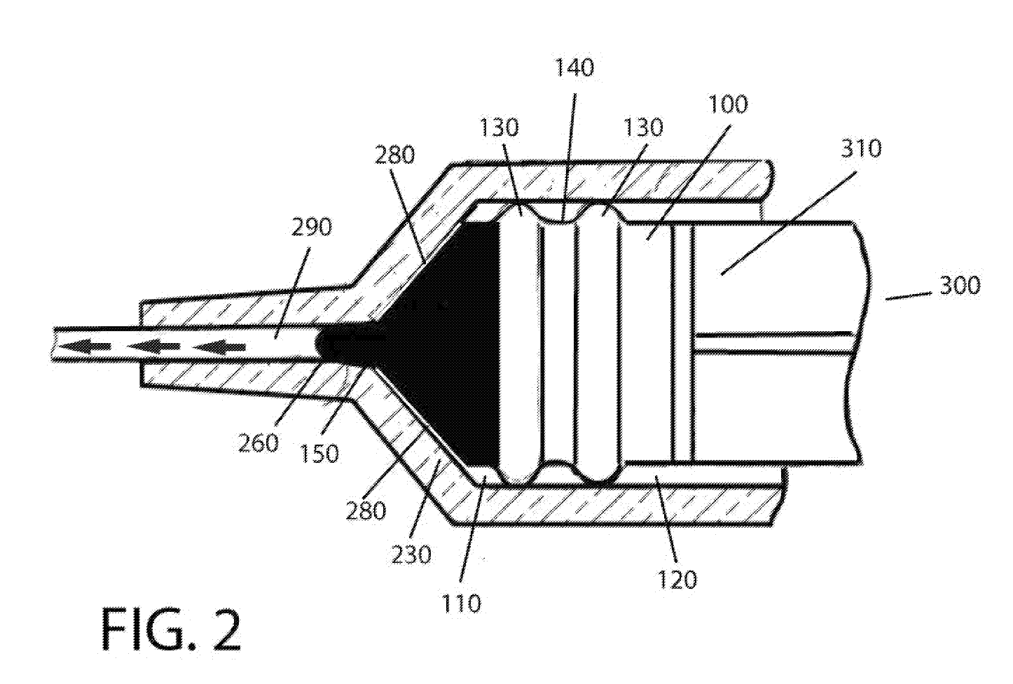

Turning now to FIGS. 1-2, the malleable (i.e., shape changing) syringe stopper 100 of the claimed invention for use in a syringe barrel 200 has a substantially cylindrical body. The syringe stopper 100 comprises a piston-like stopper body for slidable fluid-tight engagement inside the syringe barrel 200 of the syringe 1000. The syringe stopper 100 comprises a distal end 110 close to the syringe tip 210 and a proximal end 120. The syringe stopper 100 also comprises annular ribs 130 with a recess 140 therebetween. The annular ribs 130 are intended to provide a stable fluid-tight seal between the syringe stopper 100 and the syringe barrel 200.

In accordance with an exemplary embodiment of the claimed invention, a distally directed generally conically-shaped malleable protrusion 150 is located on the distal end 110 of the syringe stopper 100. The syringe stopper 100 of the claimed invention can be used in any syringe assembly, as long as it is the last stopper. That is, the syringe stopper 100 of the claimed invention is the stopper which contacts the distal end 230 of the syringe barrel 200, as shown in FIG. 2. It is appreciated the syringe stopper 100 can be a floating stopper which is not connected to a plunger rod 300. For simplicity, the claimed malleable or shape changing stopper 100 will be discussed herein in the context of a single stopper syringe as depicted in FIGS. 1-2. The syringe assembly comprises a plunger rod 300 having a distal end 310 and a proximal end 320. In accordance with an exemplary embodiment of the claimed invention, the distal end 310 of the plunger rod 300 is connected to syringe stopper 100 through the action of a threaded tip (not shown) on the distal end 310 of the plunger rod 300 which engages a threaded recess (not shown) in the syringe stopper 100. It is appreciated that there are many ways to join the plunger rod 300 to the syringe stopper 100 and threaded engagement is one of many possibilities. In accordance with an aspect of the claimed invention, the syringe stopper 100 can include a cavity in the proximal end 120 of the syringe stopper 100 shaped to accept the distal end 310 of the plunger rod 300. Preferably, the plunger rod 300 has an enlarged distal end 310 so that the syringe stopper 100 and the plunger rod 300 come together in a snap-fit arrangement. Alternatively, the syringe stopper 100 can be adhesively attached to the plunger rod 300. In accordance with another aspect of the claimed invention, the plunger rod 300 and the syringe stopper 100 can be integrally molded wherein a soft thermoplastic elastomer, which is preferably malleable when cured, is injected into one end to form the syringe stopper 100 and a more rigid plastic is injected on the other end to form the plunger rod 300.

The syringe barrel 200 comprises an open proximal end 220, distal end 230 and a cylindrical body portion 240 therebetween defining a chamber 250 for retaining liquid 400, preferably liquid medication. The syringe barrel 200 can be made of glass, plastic or the like. As shown in FIGS. 1-2, the syringe stopper 100 is positioned in fluid-tight engagement inside the syringe barrel 200 through the action of annular ribs 130 with the distal end 110 of the syringe stopper 100 facing the distal end 230 of the syringe barrel 200. The distal end 230 of the syringe barrel 200 includes a passageway 260 which is in fluid communication with chamber 250. An elongate needle cannula 500 comprises a distal end 510, a proximal end 201 and a bore therethrough. The proximal end 520 of the needle cannula 500 is connected, preferably removeably connected, to the distal end 230 of the syringe barrel 200 so that the bore of the needle cannula 500 is in fluid communication with the chamber 250 through the passageway 260. It is appreciated that the needle cannula 500 can be permanently attached to the syringe barrel 200, removably or frictionally attached to the syringe tip 210 of the syringe barrel 200.

In mass production, uniformity in the shape of the interior area 270 of the syringe barrel 200 at the distal end 230 may be difficult to control. The syringe stopper 100 of the claimed invention is made of durable and malleable material, e.g. natural rubber, synthetic rubber, high density polyethylene, neoprene, thermoplastic elastomers, silicone, latex and the like, to conform to any shape of the interior area 270 of the syringe barrel 200 to eliminate any dead space to expel the liquid 400 from the syringe barrel 200, thereby minimizing the potential for liquid 400 being trapped in the syringe barrel 200. As shown in FIG. 2, the claimed malleable stopper 100 changed its shape to occupy the dead space 280 of the syringe barrel 200. Although the shape of the barrel interior area 270 may not be precisely uniform from syringe barrel 200 to syringe barrel 200, the claimed malleable syringe stopper 100 can change its shape or contour to conform the non-uniform barrel interior area 270 of the syringes 1000. In accordance with an aspect of the claimed invention, the claimed malleable stopper 100 expands to occupy any dead space 280 in the syringe barrel 200 including interior area 290 of the syringe tip 210, thereby minimizing the potential for liquid 400 being trapped in the syringe tip 210 and the syringe barrel 200.

Various omissions, modifications, substitutions and changes in the forms and details of the device illustrated and in its operation can be made by those skilled in the art without departing in any way from the spirit of the present invention. Accordingly, the scope of the invention is not limited to the foregoing specification, but instead is given by the appended claims along with their full range of equivalents.

1. A syringe stopper for use in a syringe having a substantially cylindrical syringe barrel, comprising a piston-like plunger rod for sliding inside the syringe barrel in a fluid-tight engagement, and a malleable stopper on and protruding outwardly from a distal end of said plunger rod and changing shape in response to a pressure applied thereon.

2. The syringe stopper of claim 1, wherein the plunger rod and an angled wall of the syringe barrel applies the pressure on said malleable stopper.

3. The syringe stopper of claim 2, wherein said malleable stopper changes shape to occupy dead spaces in the syringe barrel when the pressured is applied thereon by the plunger rod and the angled wall of the syringe barrel.

4. The syringe stopper of claim 2, wherein said malleable stopper changes shape to occupy dead spaces in the syringe barrel and an interior area of the syringe tip when the pressured is applied thereon by the plunger rod and the angled wall of the syringe barrel.

5. The syringe stopper of claim 1, wherein said malleable stopper is made of durable and shape changing material.
